(19) United States
(12) Patent Application Publication
(10) Pub. No.: US 2022/0096333 A1
(43) Pub. Date: Mar. 31, 2022

(54) DRY POWDER COMPOSITIONS, METHOD OF USING THE SAME

(71) Applicant: ISP INVESTMENTS LLC, Wilmington, DE (US)

(72) Inventors: David C. STREULI, Wayne, NJ (US); Anthony LUSCHEN, Wayne, NJ (US); Panagiota ANTONOPOULOS, Emerson, NJ (US)

(73) Assignee: ISP INVESTMENTS LLC, Wilmington, DE (US)

(21) Appl. No.: 17/430,158

(22) PCT Filed: Feb. 11, 2020
(86) PCT No.: PCT/US2020/017623

§ 371 Date: Aug. 11, 2021

Related U.S. Application Data
(60) Provisional application No. 62/803,651, filed on Feb. 11, 2019.

(51) Int. Cl. A61K 8/02; A61K 8/81; A61Q 5/02; A61Q 19/10

(52) U.S. Cl. CPC A61K 8/022; A61K 8/8182; A61K 2800/412; A61Q 19/10; A61Q 5/02

(57) ABSTRACT

A dry powder composition for treating hair or skin comprising crosslinked polyvinyl pyrrolidone (PVP) having an average particle size of 15 to 120 μm is disclosed. Also disclosed is the application of the dry powder composition in personal care, laundry care, home care, pet care and industrial care.

### FIELD OF THE INVENTION

The present application relates to a dry powder composition. More particularly, the application relates to a dry powder composition comprising crosslinked polyvinyl pyrrolidone (PVP) and a method of treating human/animal hair skin using the dry powder composition.

### BACKGROUND OF THE INVENTION

Regular washing of hair tresses is carried out with conventional liquid or gel shampoo followed by rinsing with water, and frequent hair washes require large amounts of water. Washing of hair, hair tresses or skin becomes difficult in the absence or scarcity of water and regular washing of hair or skin with water can make hair or skin dry and damaged. As an alternative, a dry shampoo can be used. When dry shampoo is sprayed onto the hair or skin, the carrier evaporates, the dust or sebum on hair or skin is absorbed by the powder, and the powder is removed simply by brushing or blotting from the scalp or gently rubbing the skin. This saves water, is more convenient for the user, and provides cleaner and better feeling hair and skin for the consumer.

U.S. Pat. No. 9,801,793 assigned to HERB UK Ltd. discloses a product comprising a dry shampoo composition and a spray bottle comprising Oryza sativa (Rice) hull powder; smectite clay, tapioca starch, corn starch and potato starch.

French Patent FR2549369 assigned to Behot Francois discloses polyvinyl pyrrolidone (PVP) based dry shampoo compositions wherein the polyvinyl pyrrolidone has particle size range between 2 and 8 microns.

U.S. Pat. No. 5,872,087 assigned to Gem Energy Industry Ltd. Discloses an herbal dry shampoo composition comprising extracts of Cocoa nucifera, Hibiscus rosa sinesis, Sapindus trifoliatus, Trigonella foenum graeceum and Bassia malabarica.

The use of alumina and activated charcoal in dry shampoo powders has been observed. However, they are ineffective. Drawbacks include residue left over, sooty appearance, sticky nature and the like.

In view of the foregoing, there still exists a need in the pertinent art for an effective dry powder shampoo which: (i) helps to remove oil and sebum from hair tresses, (ii) is non-hazardous and convenient for users, including bedridden patients and travelers and (iii) can maintain the lubricant nature of hair tresses.

### SUMMARY OF THE INVENTION

Applicants have now surprisingly found that dry shampoo formulated with crosslinked polyvinyl pyrrolidone (PVP) having a specific particle size applied to the hair or skin effectively eliminates sebum from hair or skin, and thereby provides a simple and effective cleaning method which is completely substantially free of water.

One aspect of the present application is to provide a cleaning composition, particularly a dry powder composition, for treating hair and skin comprising crosslinked polyvinyl pyrrolidone (PVP) having an average particle size of 15 to 120 μm.

Another aspect of the present application is to provide a dry powder composition for treating hair and skin comprising crosslinked polyvinyl pyrrolidone (PVP) and water in the ratio of from 1:99 to 20:80; wherein the crosslinked polyvinyl pyrrolidone (PVP) has an average particle size range of from 15 to 120 μm.

Yet another aspect of the present application is to provide a dry powder composition for treating hair and skin comprising: (i) 0.1 wt. % to 10 wt. % of crosslinked polyvinyl pyrrolidone (PVP) having an average particle size of 15 to 120 μm; (ii) 0.0 wt. % to 99 wt. % of at least one alcohol; (iii) 0.0 wt. % to 99 wt. % of water; (iv) 0.0 wt. % to 20 wt. % of at least one glycol; (v) 0.0 wt. % to 10 wt. % of at least one cationic; anionic, amphoteric, or non-ionic polymer; and (vi) optionally, 0.1 wt. % to 90 wt. % of at least one compound selected from hydrocarbon, dimethyl ether, hydrofluorocarbon, hydrofluoric-olefin, and compressed gas including air, nitrogen, carbon dioxide, and nitrous oxide.

Yet another aspect of the present application is to provide a method of cleaning human hair or hair tresses comprising contacting the hair tresses with a dry powder composition containing crosslinked polyvinyl pyrrolidone (PVP) having an average particle size of 15 to 120 μm.

Another aspect of the present application is to provide a method of reducing the soil or preventing the soil deposition on hair or skin, wherein the method comprises contacting the human hair with a dry powder composition comprising crosslinked polyvinyl pyrrolidone (PVP) having an average particle size of 15 to 120 μm.

Still another aspect of the present application is to provide a method of treating acne and providing aesthetic feel to the skin, wherein the method comprises using a dry powder composition comprising crosslinked polyvinyl pyrrolidone (PVP) having an average particle size of 15 to 120 μm.

Yet another aspect of the present application is to provide a method of treating pet(s) or animal hair, wherein the method comprises contacting the pet or animal hair with a dry powder composition comprising crosslinked polyvinyl pyrrolidone (PVP) having an average particle size of from 15 to 120 μm.

Yet another aspect of the present application is to provide a method of treating a scalp, wherein the method comprises contacting the scalp with a dry powder composition comprising crosslinked polyvinyl pyrrolidone (PVP) having an average particle size of from 15 to 120 μm, wherein the scalp can be human or animal scalp.

Yet another aspect of the present application is to provide a method of treating body odor and body oil, the method comprises contacting the human skin with a dry powder composition comprising crosslinked polyvinyl pyrrolidone (PVP) having an average particle size of from 15 to 120 μm.

Yet another aspect of the present application is to provide a method of preparing personal hygiene products, the method comprises preparing the personal hygiene product with a dry powder composition comprising crosslinked polyvinyl pyrrolidone (PVP) having an average particle size of from 15 to 120 μm.

Yet another aspect of the present application is to provide a method of use in fragrance retention or organic dyeing retention, the method comprises combining the fragrance or organic dye with a dry powder composition comprising crosslinked polyvinyl pyrrolidone (PVP) having an average particle size of from 15 to 120 μm.

### BRIEF DESCRIPTION OF DRAWINGS

For a fuller understanding of the nature and advantages of the present invention, reference should be had to the following detailed description taken in connection with the accompanying drawings, in which:

FIG. 1 shows graphically negative performance of comparative samples having D50 of 4 μm-11 μm—Shine, combing, residue and cleaning/manageability compared to the dry powder composition of this invention having D50 of 15 μm-25 μm.

FIG. 2 shows pictorially performance on hair—commercial dry shampoo with starch vs. current dry powder containing crosslinked polyvinyl pyrrolidone (PVP).

FIG. 3 shows graphically performance on hair—% change from untreated state as a function of red, black, and light background and color intensity measurements.

FIG. 4 shows graphically sorption (Adsorption and/or Absorption) analysis.

FIG. 5 shows pictorially acoustical analysis of combing across a wide frequency range for commercial dry shampoo vs. current aerosol spray containing crosslinked polyvinyl pyrrolidone (PVP).

### DETAILED DESCRIPTION OF THE INVENTION

Before explaining embodiment(s) of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting. The following detailed discussion of the various alternative and preferred embodiments will illustrate the general principles of the invention. The following detailed description is intended to be representative only and not limiting as to the described inventive elements. Many variations can be derived by one skilled in the art which are included within the scope of the present invention.

Unless otherwise defined herein, technical terms used in connection with the present disclosure shall have the meanings that are commonly understood by those of ordinary skill in the art. Further, unless otherwise required by context, singular terms shall include pluralities and plural terms shall include the singular.

All percentages, parts, proportions and ratios as used herein, are by weight of the total composition, unless otherwise specified. All such weights as they pertain to listed ingredients are based on the active level and, therefore; do not include solvents or byproducts that can be included in commercially available materials, unless otherwise specified.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All references to singular characteristics or limitations of the present disclosure shall include the corresponding plural characteristic or limitation, and vice-versa, unless otherwise specified or clearly implied to the contrary by the context in which the reference is made.

As utilized in accordance with the present disclosure, the following terms, unless otherwise indicated, shall be understood to have the following meanings.

The use of the word “a” or “an” when used in conjunction with the term “comprising” may mean “one,” but it is also consistent with the meaning of “one or more, at least one,” and “one or more than one.” The use of the term “or” is used to mean “and/or” unless explicitly indicated to refer to alternatives only if the alternatives are mutually exclusive, although the disclosure supports a definition that refers to only alternatives and “and/or.” Throughout this application, the term “about” is used to indicate that a value includes the inherent variation of error for the quantifying device, the method being employed to determine the value, or the variation that exists among the study subjects. For example, but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first”, “second”, “third”, “fourth”, etc.) is solely for the purpose of differentiating between two or more items and, unless otherwise stated, is not meant to imply any sequence or order or importance to one item over another or any order of addition.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “or combinations thereof” as used herein refers to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Numerical ranges as used herein are intended to include every number and subset of numbers contained within that range, whether specifically disclosed or not. Further, these numerical ranges should be construed as providing support for a claim directed to any number or subset of numbers in that range.

The term “PVP” as used herein refers to polyvinyl pyrrolidone”.

The term “polyol” as used herein refers to any organic compound(s) that comprises “at least two hydroxyl” functionalities, wherein, the organic compound may be aliphatic, aromatic, heterocyclic, or alicyclic in nature.

The terms “preferred” or “preferably” and variants as used herein, refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

Applicants have surprisingly discovered that crosslinked polyvinyl pyrrolidone (PVP) having a specific particle size range results in an effective dry powder composition for various end-user applications.

In one embodiment, the present application provides a dry powder composition for treating hair and skin comprising crosslinked polyvinyl pyrrolidone (PVP) having an average particle size range of from 15 to 120 μm. In another embodiment, the particle size range is from 15 to 100 μm. In other embodiments, the particle size range is from 15 to 60 μm or from 15 to 25 μm

According to one important embodiment of the present application, the crosslinked polyvinyl pyrrolidone (PVP) is present in an amount of from 0.1 wt. % to 99 wt. % of the total composition. In another embodiment, the crosslinked polyvinyl pyrrolidone (PVP) is present in an amount of 0.25 wt. % to 10 wt. % of the total composition.

The dry powder composition of the present application may further comprise at least one glycol(s) selected from the group consisting of glycerin, diglycerin, polyethylene glycol, propylene glycol, butylene glycol, hexylene glycol, caprylyl glycol, polyol and combinations thereof.

According to another embodiment, the dry powder composition can be used in personal care, laundry, home care, pet care and industrial care applications.

In another embodiment, the personal care composition of the present application is a leave-on volumizing or rinse-out hair conditioning or hair styling composition.

In another embodiment, the present application provides a dry powder composition for treating hair and skin that comprises (i) crosslinked polyvinyl pyrrolidone and (ii) water in the ratio of from 1:99 to 20:80; wherein the crosslinked polyvinyl pyrrolidone (PVP) has an average particle size range of from 15 to 120 μm.

In another embodiment, the crosslinked polyvinyl pyrrolidone (PVP) present in the dry powder composition of the present application can advantageously swell in presence of water and can improve the hair tress cleaning and soil deposition reduction, and wherein, the amount of crosslinked polyvinyl pyrrolidone (PVP) is in the range of from 2 to 10% of the total volume.

In another embodiment, the present application provides a dry powder composition for treating hair and skin comprising: (i) from 0.1 wt. % to 10 wt. % of crosslinked polyvinyl pyrrolidone (PVP) having an average particle size range of 15 to 120 μm; (ii) from 0.0 wt. % to 99 wt. % of at least one alcohol; (iii) from 0.0 wt. % to 99 wt. % of water; (iv) from 0.0 wt. % to 20 vi. % of at least one glycol; (v) from 0.0 wt. % to 10 wt. % of at least one cationic; anionic, amphoteric, or non-ionic polymer(s); and (vi) optionally, from 0.1 wt. % to 90 wt. % of at least one compound selected from the group consisting of hydrocarbon, a dialkyl ether, a hydrofluorocarbon, a hydrofluoro-olefin, and a compressed gas that includes air, nitrogen, carbon dioxide, and nitrous oxide.

In another embodiment, the alcohol is selected from the group consisting of ethanol, phenoxyethanol, aminomethyl propanol, propylene glycol, caprylyl glycol, butylene glycol, behenyl alcohol, lauryl alcohol, myristyl alcohol, cetyl alcohol and combinations thereof.

In another embodiment, the suitable ester(s) employed in the current application include octenylsuccinate or its structurally similar analogues.

According to another embodiment, the hydrocarbon employed in the current application can be hydrocarbon (A-40), hydrocarbon (A-46), hydrofluoro carbon-152A, microcrystalline wax, and or petrolatum.

Suitable cationic polymers employed in the current application include, but are not limited to, acrylamide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Cationic polymers that can be used include cationic guar gum derivatives, such as guar hydroxypropyltrimonium chloride (commercially available from Celanese Corp. in their Jaguar R series). Other materials include quaternary nitrogen-containing cellulose ethers (e.g., as described in U.S. Pat. No. 3,962,418, incorporated herein by reference), and copolymers of etherified cellulose and starch (e.g., as described in U.S. Pat. No. 3,958,581, incorporated herein by reference).

In another embodiment, the present application provides for the use of the dry powder composition in skin care, hair care, animal care, and industrial care applications.

In another embodiment, the present application discloses a dry powder composition used in end-user based personal care applications further comprising at least one personal care additive selected from the group consisting of preservatives, antioxidants, chelating agents, sunscreen agents, proteins, amino acids, vitamins, dyes, hair coloring agents, plant extracts, humectants, fragrances, emollients, lubricants, penetrants, thickeners, viscosity modifiers, polymers, resins, hair fixatives, film formers, surfactants, detergents, emulsifiers, pacifying agents, volatiles, propellants, liquid vehicles, carriers, salts, pH adjusting agents, neutralizing agents, hair conditioning agents, anti-static agents, anti-frizz agents, anti-dandruff agents, hair waving agents, relaxers, absorbents, fatty substances, gelling agents, moisturizers, hydrophilic or lipophilic active agent, fillers, reducing agents, cosmetic oils, perfumes, liquid vehicles, solvents, and combinations thereof.

In another embodiment, suitable conditioning agents that are useful for conditioning and/or moisturizing the hair tresses include, but are not limited to, lauric acid, silicones selected from phenyl trimethicones, dimethicones, cyclomethicones, and amino silicones.

According to one aspect of the present application, the dry powder composition is a dry powder shampoo composition. The shampoo composition may comprise blends including corn, aluminumstarch octenylsuccinate, tapioca starch, polymethyl silsesquioxane, rice starch, acrylates copolymer, and magnesium carbonate.

According to one embodiment, the composition is formulated into a mousse, a spray, a gel, a cream, a lotion, a paste, a powder or as a rinse-off product.

In yet another embodiment, the present application provides a method of cleaning human hair comprising contacting the hair tresses with a dry powder composition containing crosslinked polyvinyl pyrrolidone (PVP) having an average particle size range of from 15 to 120 μm. The non-limiting hair tresses are Caucasian, Asian, or African origin selected from fine, medium and coarse virgin, damaged, colored, grey or bleached hairs and wherein, the hair tress has textures comprising wavy, curly, oily or kinky pattern, and wherein, the method is used for damp hair or for dry hair.

In yet another embodiment, the present application provides a method of reducing soil or preventing soil deposition on hair or skin, the method comprising contacting the hair with a dry powder composition comprising crosslinked polyvinyl pyrrolidone (PVP) having an average particle size range of from 15 to 120 μm. The dry powder can be sprayed onto the hair directly or can be sprinkled into user's hand and then applied to the hair or skin. The composition after applying can be equally distributed through the hair or skin by massaging into the hair or skin, for example, in order to remove grease, oil and other undesirable elements from soiled hair or skin. Any excess or residue of shampoo can be removed from the hair or skin by combing or simply brushing the hair or skin. The composition provides improved cleaning, combing, shine, manageability, and has less residual impact.

In yet another embodiment, the present application provides a method of treating acne and providing aesthetic feel to the skin, the method comprising using a dry powder composition comprising crosslinked polyvinyl pyrrolidone (PVP) having an average particle size of from 15 to 120 μm.

Accordingly, acne refers to skin disorder wherein, inflammation occurs affecting the sebaceous glands. Acne lesions primarily involve the sebaceous glands located on the face, neck, chest and back. Both closed comedones (blackheads) and open comedones (whiteheads) are caused by hyperkeratinization of the infundibulum of the sebaceous duct. These keratinous plugs block the flow of sebum. The inventive method involves applying the dry powder composition onto the affected part of skin as a coating or dressing or as a filler.

In yet another embodiment, the present application provides a method of treating animal hair or human hair, the method comprising contacting the animal/human hair with a dry powder composition comprising crosslinked polyvinyl pyrrolidone (PVP) having an average particle size of from 15 to 120 μm, wherein the animal hair is pet hair.

In yet another embodiment, the present application provides a method of treating scalp, wherein the method comprising contacting the scalp with a dry powder composition comprising crosslinked polyvinyl pyrrolidone (PVP) having an average particle size of from 15 to 120 μm, wherein the scalp is human scalp or animal scalp.

In yet another embodiment, the present application provides a method of treating body odor and body oil, the method comprising contacting the human skin with a dry powder composition comprising crosslinked polyvinyl pyrrolidone (PVP) having an average particle size of from 15 to 120 μm.

Accordingly, body oil refers to microorganisms and odors which adhere to skin and interact with oil-containing composition, and that they are not effectively removed by water and soap. The current method employs removing such oils and odors caused by them.

In yet another embodiment, the present application provides a method of preparing personal hygiene products, the method comprising preparing the personal hygiene product with a dry powder composition comprising crosslinked polyvinyl pyrrolidone (PVP) having an average particle size of from 15 to 120 μm.

Accordingly, personal hygiene product refers to any material which prevents seepage onto the skin, clothing or external environment. Personal hygiene products include tampons; bed pads; disposable adult diapers; disposable adult briefs; disposable sanitary napkins, sanitary napkins with adhesive strips and wings; and panty liners. Most people will at some point in their life use a personal hygiene product for a period of time.

In yet another embodiment, the present application provides a method of use in fragrance retention or organic dyeing retention, the method comprising combining the fragrance or organic dye with a dry powder composition comprising crosslinked polyvinyl pyrrolidone (PVP) having an average particle size of from 15 to 120 μm.

Accordingly, fragrance retention refers to any fixative property or substance in association with a fragrance, perfume or scent, expressed in levels of perceived fragrance intensity or effect after specific time intervals, as measured by a panel made up of experienced fragrance © valuators.

In yet another embodiment, the present application provides a dry powder composition for treating “non-keratinous fibers” comprising crosslinked polyvinyl pyrrolidone (PVP) having an average particle size of from 15 to 120 μm. The term “non-keratinous fibers” refer to fibrous structures devoid of keratin (a fibrous protein forming the main structural constituent of hair, feathers, hoofs, claws, horns, and the like). These fibrous structures can be staple length fibers or continuous fibers, and can be natural fibers such as cotton, silk, and mixtures thereof, or synthetic fibers such as polyacrylonitrile, nylon, polyamide, and polyesters, triacetate, polyethylene, propylene, and mixtures thereof, or any combinations of the natural and synthetic fibers.

The following examples illustrate the present disclosure, parts and percentages being by weight, unless otherwise indicated. Each example is provided by way of explanation of the present disclosure, not limited to the present disclosure. In fact, it will be apparent to those skilled in the art that various modifications and variations can be made in the present disclosure without departing from the scope or spirit of the invention. For instance, features illustrated or described as part of one embodiment, can be used on another embodiment to yield a still further embodiment. Thus, it is intended that the present disclosure covers such modifications and variations as come within the scope of the appended claims and their equivalents.

These examples are presented herein for purposes of illustration of the present disclosure and are not intended to be limiting, for example, the dry powder composition(s) and method of using these compositions in various end-user applications.

### EXAMPLES

EXAMPLE 1: A Dry Shampoo Paste

| Ingredients | Paste (wt. %) |
|---|---|
| Water | 59.00 |
| PVP* | 20.00 |
| Glycerin | 20.00 |
| Phenoxyethanol and caprylyl glycol | 1.00 |
| Total | 100.00 |

PVP* - dry powder crosslinked containing polyvinyl pyrrolidone available from Ashland (Advantage ™ Revive) having an average particle size of 15-25 μm.

A dry shampoo paste was prepared by adding water, glycerin, phenoxyethanol and caprylyl glycol to the main vessel and mixing. PVP was added and mixed until the mixture was distributed uniformly.

EXAMPLE 2: A Dry Shampoo Gel

| Ingredients | Gel (wt. %) |
|---|---|
| Water | 96.24 |
| PVP* | 1.00 |
| VP/AA crosspolymer | 0.85 |
| Aminomethyl propanol | 0.41 |
| Glycerin and Glyceryl Acrylate/Acrylic | 0.50 |
| Acid Copolymer and Propylene Glycol and | |
| PVM/MA Copolymer | |
| Phenoxyethanol and caprylyl glycol | 1.00 |
| Total | 100.00 |

Water was added to the main vessel and mixing was begun. PVP was added and mixed continuously until the contents were mixed uniformly. The VP/AA crosspolymer was added and mixed until the contents were completely dissolved. Remaining ingredients (aminomethyl propanol, glycerin and glyceryl acrylate/acrylic acid copolymer and propylene glycol and PVM/MA copolymer, phenoxyethanol and caprylyl glycol) were mixed individually and mixing was carried out until a uniform mixture was obtained.

EXAMPLE 3: Anhydrous Aerosol Spray

| Contents | Amount (% w/w) | | | | | | |
|---|---|---|---|---|---|---|---|
| Ethanol | 35.00 | 34.50 | 32.50 | 35.00 | 35.00 | 34.00 | 12.30 |
| PVP* | 5.00 | 5.00 | 7.50 | 2.50 | 0.75 | 3.00 | 2.50 |
| Derivatives/blends* | 0.00 | 0.00 | 0.00 | 2.50 | 2.50 | 0.00 | 0.00 |
| Disteardimonium | 0.00 | 0.00 | 0.00 | 0.00 | 0.10 | 0.00 | 0.00 |
| Hectorite or bentonite | | | | | | | |
| Silica (colloid or | 0.00 | 0.00 | 0.00 | 0.00 | 1.75 | 0.00 | 0.00 |
| fumed) | | | | | | | |
| Zeolite | 0.00 | 0.00 | 0.00 | 0.00 | 0.00 | 3.00 | 0.00 |
| Glycerin | 0.00 | 0.25 | 0.00 | 0.00 | 0.00 | 0.00 | 0.00 |
| Caprylyl glycol | 0.00 | 0.250 | 0.00 | 0.00 | 0.00 | 0.00 | 0.200 |
| Hydrocarbon (A-46) | 60.00 | 60.00 | 60.00 | 60.00 | 59.90 | 0.00 | 85.00 |
| Total | 100.00 | 100.00 | 100.00 | 100.00 | 100.00 | 100.00 | 100.00 |

Derivatives/blends* - (corn, aluminumstarch octenylsuccinate, tapioca, starch, (and) polymethylsilsesquioxane, rice starch, aluminumstarch, charcoal, Octenylsuccinate (and) acrylates copolymer (and) magnesium carbonate)

Ethanol was added to the main vessel followed by mixing. PVP was added to ethanol until mixture was uniformly distributed. Remaining ingredients (derivatives, Disteardimonium hectorite or bentonite, silica, glycerin, and caprylyl glycol were added individually until uniform mixture was obtained after each addition. The product so obtained was filled into cans and charged with propellant. An anhydrous aerosol spray was thus obtained.

EXAMPLE 4: Anhydrous Aerosol Spray

| Ingredients | Amount (% w/w) |
|---|---|
| Ethanol | 29.00 |
| PVP* | 5.00 |
| Caprolactam/PVP/dimethylaminoethyl | 5.40 |
| methacrylate | |
| Glycerin | 0.25 |
| Caprylyl glycol | 0.25 |
| Hydrocarbon (A-46) | 60.00 |
| Total | 100.00 |

Ethanol was added to the main vessel followed by mixing. Dry powder containing PVP was added to ethanol until mixture was uniformly distributed. Remaining ingredients (caprolacam/PVP/dimethylaminoethyl methacrylate, glycerin, and caprylyl glycol were added individually until uniform mixture was obtained after each addition; the product so obtained was filled into cans and charged with propellant. An anhydrous aerosol spray was thus obtained.

EXAMPLE 5: Low Water Containing Aerosol Spray

| Ingredients | Amount (% w/w) |
|---|---|
| Ethanol | 27.00 |
| Water | 10.00 |
| PVP* | 2.500 |
| Glycerin | 0.500 |
| Dimethyl ether | 60.00 |
| Total | 100.00 |

el and mixed well. Dry powder containing PVP was added and mixed until uniformly distributed. Remaining ingredients (glycerin) were added individually with mixing until uniform after each addition. Product so obtained was filled into cans and propellant was charged to obtain low water containing aerosol spray.

EXAMPLE 6: Non-Ethanol Containing Aerosol Spray

| Ingredients | Amount (% w/w) |
|---|---|
| Ethanol | 0.00 |
| Water | 68.09 |
| PVP* | 1.50 |
| Guar Hydroxypropyltrimonium chloride | 0.15 |
| Lactic acid | 0.01 |
| Glycerin | 0.25 |
| Dimethyl ether | 30.00 |
| Total | 100.00 |

Water was added to the main vessel and mixed well. PVP was added and mixed until uniformly distributed. Remaining ingredients (guar hydroxypropyltrimonium chloride, lactic acid, glycerin) were added individually with mixing until uniform after each addition. Product so obtained was filled into cans and propellant was charged to obtain non-ethanol containing aerosol spray.

EXAMPLE 7: Aerosol Foam

| Ingredients | Amount (% w/w) |
|---|---|
| Ethanol | 0.00 |
| Water | 89.14 |
| PVP* | 2.00 |
| Guar Hydroxypropyltrimonium chloride | 0.75 |
| Cetrimonium Chloride | 0.25 |
| Lactic acid | 0.03 |
| Quaternium-22 | 0.50 |
| PPG-2 Hydroxyethyl Cocamide | 0.75 |
| Phenoxyethanol and caprylyl glycol | |
| Glycerin | 0.50 |
| Hydrocarbon (A-40) | 6.00 |
| Total | 100.00 |

Water was added to the main vessel and began mixing. PVP was added and mixed until uniformly distributed. Remaining ingredients (guar hydroxypropyltrimonium chloride, Cetrimonium chloride, lactic acid, quaternium-22, PPG-2 hydroxyethyl cocamide, phenoxyethanol and caprylyl glycol, and glycerin were added individually with mixing until uniform after each addition. The product was filled into cans and charged with propellant to obtain aerosol foam.

EXAMPLE 8: Leave on Volumizing

| | Amount | Amount |
|---|---|---|
| Ingredients | (% w/w) | (% w/w) |
| Water | 83.56 | 78.61 |
| PVP* | 0.50 | 0.25 |
| Ethyl ester of PVM/MA copolymer (50% in | 0.00 | 5.00 |
| ethanol) | | |
| Aminomethyl propanol (95%) | 0.00 | 0.46 |
| guar hydroxypropyltrimonium chloride | 0.35 | 0.50 |
| polyimide I (30% in water) | 6.00 | 3.33 |
| Cetrimonium chloride | 0.20 | 0.00 |
| lactic acid | 0.04 | 0.05 |
| PPG-2 Hydroxyethyl Cocamide | 0.15 | 0.00 |
| Oleth 20 | 0.00 | 0.40 |
| amodimethicone/Cetrimonium chloride/ | 0.00 | 0.50 |
| trideceth-10 | | |
| dicetyldimonium chloride | 0.00 | 0.15 |
| Phenoxyethanol and caprylyl glycol | 0.75 | 0.50 |
| glycerin/glyceryl acrylate/acrylic acid | 0.00 | 0.25 |
| copolymer | | |
| Dimethyl Ether | 0.00 | 6.67 |
| Hydrofluorocarbon 152a | 2.45 | 0.00 |
| Hydrocarbon (A-40) | 6.00 | 0.00 |
| Hydrocarbon (A-46) | 0.00 | 3.33 |
| Total | 100 | 100 |

Water was added to the main vessel and began mixing. PVP was added and mixed until uniformly distributed. Remaining ingredients (ethyl ester of PVM/MA copolymer (50% in ethanol), aminomethyl propanol (95%), guar hydroxypropyltrimonium chloride, polyimide 1 (30% in water), cetrimoniunm chloride, lactic acid, PPG-2 hydroxyethyl cocamide, oleth 20, amodimethicone/cetrimonium chloride/trideceth-10, dicetyldimonium chloride, phenoxyethanol and caprylyl glycol, and glycerin glyceryl acrylate/acrylic acid copolymer were added individually with mixing until uniform after each addition. A leave-on volumizing shampoo was obtained.

Example 9: Whipped Cream

| Ingredient | Amount (wt. %) |
|---|---|
| Phase A | |
| Aqua | 55.95 |
| Tetra sodium EDTA | 0.05 |
| Glycerin | 5.00 |
| Butylene Glycol | 2.00 |
| Water/aqua (and) Glycerin (and) Glyceryl | 7.00 |
| Acrylate/Acrylic Acid Copolymer (and) | |
| PVM/MA Copolymer | |
| 1,2-Hexanediol | 3.00 |
| Phase B | |
| PVP K series | 1.20 |
| Phase C | |
| Glyceryl Stearate & Behenyl Alcohol & Palmitic | 0.60 |
| Acid & Stearic Acid & Lecithin & Lauryl | |
| Alcohol & Myristyl Alcohol & Cetyl Alcohol | |
| Jojoba Esters (and) Acacia Decurrens Flower Wax | 1.00 |
| (and) Helianthus Annuus (Sunflower) Seed Wax | |
| (and) Polyglycerin-3 | |
| Phenoxyethanol (and) Caprylyl Glycol | 1.00 |
| Isodecyl Neopentanoate | 4.00 |
| Diheptyl Succinate (and) Capryloyl Glycerin/Sebacic | 2.00 |
| Copolymer | |
| VP/acrylates/lauryl methacrylate copolymer | 0.50 |
| Phase D | |
| Sodium Polyacrylate | 1.50 |
| Phase E | |
| Alcohol | 5.00 |
| Phase F | |
| Isododecane (and) Hydrogenated | 5.00 |
| tetradecenyl/methyl pentadecene | |
| Phase G | |
| | 1.50 |
| Macrocystis Pyrifera (Kelp) Extract | 1.00 |
| Phase H | |
| Tapioca Starch (and) polymethylsilsesquioxane | 1.50 |
| polyvinylpyrrolidone (PVP)* | 0.50 |
| fragrance/perfume | 0.20 |
| Boron Nitride | 0.50 |
| Total | 100.00 |

Dry powder composition was formulated into a whipped cream. The method includes phase wise addition of different ingredients as provided in the above table.

### Example 10: Effect of Particle Size on Performance of Hair

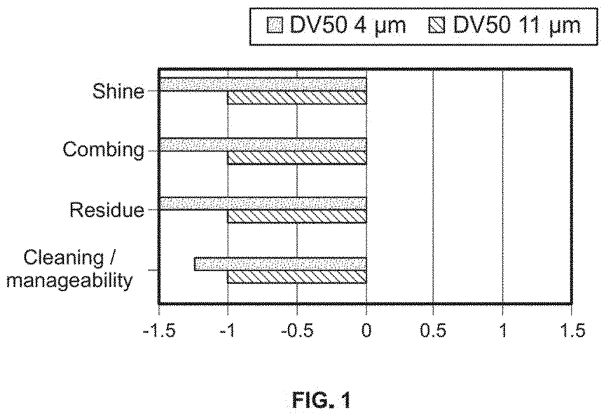

Performance of hair was tested for parameters like cleaning, residue, combing and shine. Average particle size 4 μm and 11 μm were tested as against baseline average particle size of 25 μm. Details are provided in FIG. 1. It was observed that the crosslinked PVP with particle size DV50 15-120 μm delivers improved performance over the range of 4-11 μm. This is determined through the evaluation of hair treated with sebum and then the crosslinked PVP materials. The DV50 15-120 μm range delivered less residue, improved hair color and shine and overall cleaning and manageability.

### Example 11: Comparison of Hair Performance with Commercial Sample

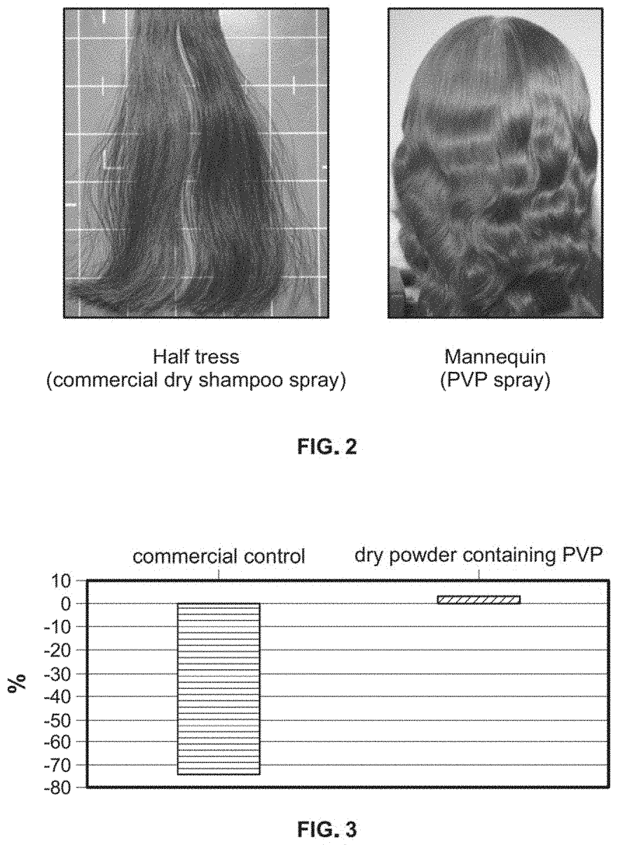

The dry powder containing PVP delivers improved cleaning and hair condition as demonstrated through less fiber clumping and residue, and improved manageability, shine and color perception. Employing a technique called hyperspectral imaging, which acquires data across the visible spectrum, a mannequin was analyzed for light background and color intensity before and after being treated with sebum and then the dry shampoo product. The dry powder containing PVP returns hair to a level of shine and color nearly at parity to the untreated (before) state. Details are provided in FIGS. 2 and 3.

### Example 12: Sorption Data

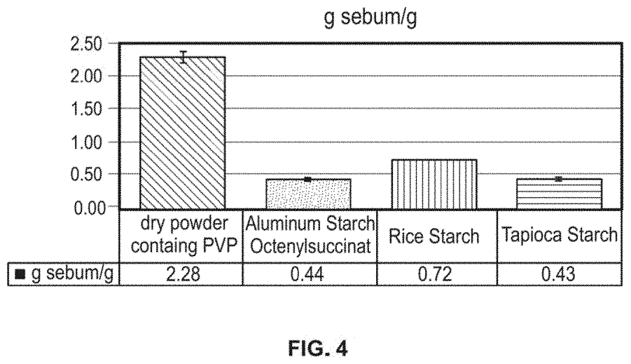

Measurements were obtained by weighing a fixed amount of material and slowly adding sebum at elevated temperature. It was observed that the mixture goes from a taffy and cohesive phase (solid-like) to a liquid-like phase with free sebum. The sorption capacity is determined by the amount of sebum sorbed prior to transitioning to the liquid-like system. PVP absorbs/adsorbs more than 4× the sebum vs the starch-based material. Details are provided in FIG. 4.

### Example 13: Combing Acoustics

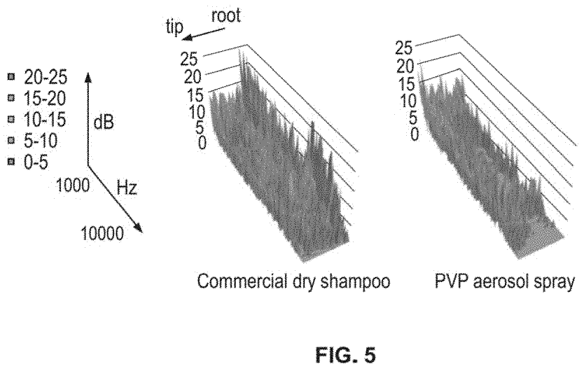

Acoustical analysis of combing across a wide frequency range of typical human hearing demonstrates dry powder containing PVP to deliver lower decibels and less frequency range than a commercial control containing the starch-based material—improved fiber alignment and less residue/drag through the hair were observed. Details are provided in FIG. 5.

The above detailed description relates to various embodiments of the technology but is not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments applicable to a wide range of compositions. The present application includes all such improvements and modifications and is limited only by the scope of the claims.

What is claimed is:

1. A dry powder composition for treating hair and skin, the dry powder composition comprising crosslinked polyvinyl pyrrolidone (PVP) having an average particle size of from 15 to 120 μm.

2. The dry powder composition according to claim 1, wherein the crosslinked polyvinyl pyrrolidone (PVP) has an average particle size of from 15 to 100 μm.

3. The dry powder composition according to claim 1, wherein the crosslinked polyvinyl pyrrolidone (PVP) has an average particle size of from 15 to 60 μm.

4. The dry powder composition according to claim 1, wherein the crosslinked polyvinyl pyrrolidone (PVP) is present in the composition in an amount of from 0.1% to 99% of the total weight.

5. The dry powder composition according to claim 1, wherein the crosslinked polyvinyl pyrrolidone (PVP) is present in the composition in an amount of from 0.25% to 10% of the total weight.

6. The dry powder composition according to claim 1, wherein the composition further comprises a glycol selected from the group consisting of glycerin, propylene glycol, butylene glycol, caprylyl glycol, polyol and combinations thereof.

7. The dry powder composition according to claim 1, wherein the composition can be used in personal care and animal care.

8. The dry powder composition according to claim 7, wherein the personal care composition is a leave-on volumizing, rinse-out hair conditioning or hair styling composition.

9. A dry powder composition for treating hair and skin, the dry powder composition comprising crosslinked polyvinyl pyrrolidone (PVP) and water in the ratio of from 1:99 to 20:80; wherein the PVP has an average particle size of from 15 to 120 μm.

10. The dry powder composition according to claim 9, wherein the crosslinked polyvinyl pyrrolidone (PVP) swells in an amount of from 2 to 10 vol. % and improves cleaning and soil reduction in hair and skin.

11. The dry powder composition according to claim 9, wherein the composition further comprises a glycol selected from the group consisting of glycerin, propylene glycol, butylene glycol, caprylyl glycol, polyol and combinations thereof.

12. A dry powder composition for treating hair and skin, the composition comprising:
(i) from 0.1 wt. % to 10 wt. % of crosslinked polyvinyl pyrrolidone having an average particle size of 15 to 120 μm;
(ii) from 0.0 wt. % to 99 wt. % of at least one alcohol;
(iii) from 0.0 wt. % to 99 wt. % of water;
(iv) from 0.0 wt. % to 20 wt. % of at least one glycol;
(v) from 0.0 wt. % to 10 wt. % of at least one cationic, anionic, amphoteric, or non-ionic polymer; and
(vi) optionally, from 0.1 wt. % to 90 wt. % of at least one compound selected from hydrocarbons, dialkyl ether, hydrofluorocarbons, hydrofluoro-olefins, and a compressed gas including air, nitrogen, carbon dioxide, and nitrous oxide.

13. The dry powder composition according to claim 12, wherein the composition is used in a hair shampoo composition to provide improved cleaning, combing, shine, manageability, and less residual impact.

14. The dry powder composition according to claim 13, wherein the composition is formulated as a mousse, a spray, a gel, a cream, a lotion, a paste, a stick, a powder or as a rinse-off product.

15. A method of cleaning human hair or skin comprising contacting a hair tress or skin with a dry powder composition containing crosslinked polyvinyl pyrrolidone (PVP) having an average particle size of from 15 to 120 μm.

16. The method according to claim 15, wherein the hair tresses are Caucasian, Asian, or African origin selected from fine, medium and coarse virgin, damaged, colored, grey or bleached hairs and wherein, the hair tress has textures comprising wavy, curly, oily or kinky pattern.

17. The method according to claim 15, wherein the method is used for damp or dry hair.

18. A method of reducing soil or preventing soil deposition on hair or skin, the method comprises contacting the hair or skin with a dry powder composition comprising crosslinked polyvinyl pyrrolidone (PVP) having an average particle size of from 15 to 120 μm.

19. A method of treating acne and providing aesthetic feel to the skin, the method comprising contacting the skin with a dry powder composition comprising crosslinked polyvinyl pyrrolidone (PVP) having an average particle size of from 15 to 120 μm.

20. A method of treating animal hair, the method comprising contacting the animal hair with a dry powder composition comprising crosslinked polyvinyl pyrrolidone (PVP) having an average particle size of from 15 to 120 μm.

21. A method of treating scalp, the method comprising contacting the scalp with a dry powder composition comprising crosslinked polyvinyl pyrrolidone (PVP) having an average particle size of from 15 to 120 μm, wherein the scalp is human scalp or animal scalp.

22. A method of treating body odor and body oil, the method comprises contacting the human skin with a dry powder composition comprising crosslinked polyvinyl pyrrolidone (PVP) having an average particle size of from 15 to 120 μm.

23. A method of preparing a personal hygiene product, the method comprising preparing the personal hygiene product with a dry powder composition comprising crosslinked polyvinyl pyrrolidone (PVP) having an average particle size of from 15 to 120 μm.

24. A method of use in fragrance retention or organic dyeing retention, the method comprising combining the fragrance or organic dye with a dry powder composition comprising crosslinked polyvinyl pyrrolidone (PVP) having an average particle size of from 15 to 120 μm.
